(19) United States
(12) Patent Application Publication
(10) Pub. No.: US 2008/0171350 A1
(43) Pub. Date: Jul. 17, 2008

(54) Neural Cell Differentiation Method From Es Cells

(75) Inventors: Yves-Alain Barde, Basel (CH); Miriam Bibel, Basel (CH); Jens Richter, Freiburg (DE); Kerry Lee Tucker, Heidelberg (DE)

Correspondence Address: NOVARTIS;CORPORATE INTELLECTUAL PROPERTY, ONE HEALTH PLAZA 104/3, EAST HANOVER, NJ, 07936-1080, US

(73) Assignee: NOVARTIS FORSCHUNGSSTIFTUNG, ZWEIGNIEDERLASSUNG FRIEDRICH MIESCHER INSTITU FOR BIOMEDICAL RESEARCH, Basel (CH)

(21) Appl. No.: 11/568,748

(22) PCT Filed: May 4, 2005
(86) PCT No.: PCT/EP2005/004886

§ 371 Date: Mar. 28, 2008

(30) Foreign Application Priority Data
May 5, 2004 (GB) GB 0410011.1

(51) Int. Cl. C12N 5/02; C12Q 1/02

(52) U.S. Cl. 435/29; 435/377

(57) ABSTRACT

A method for inducing differentiation of embryonic stem cells into neuronal precursors is provided as well as an assay for neuronal precursor or progenitor cells and a method for identifying agents that inhibit or reduce an increase in neurite degeneration.

### FIELD OF THE INVENTION

The present invention relates to in vitro generation of neuronal precursor or progenitor cells or neurons from pluripotent cells, especially ES cells.

### BACKGROUND

It has long been known that pluripotent cells such as embryonal carcinoma (EC) cells and embryonic stem (ES) cells can be differentiated into neurons in vitro. In principle, work with ES cells creates the possibility of isolating cells at selected stages of differentiation and of characterising neuronal precursors. ES cells facilitate the study of molecular and genetic developmental pathways in vitro, and are also a potential source of cells for transplantation into the brain to treat neurological disease.

However, these and other applications have been hindered by the heterogeneous, disorganised and frequently non-reproducible nature of neuronal development in culture. Cellular heterogeneity is an enormous problem with the use of ES cells to generate neural cells (for reviews see ref. 3, 4). Typically, neuronal cultures derived from ES cells contain a variety of different neuronal subtypes as well as non-neuronal cells including glial cells. A lack of sufficiently large numbers of cells with defined and uniform phenotypes is a major difficulty in neurobiology. There has been no method for the generation of neurons from ES cells that leads to consistent numbers of neurons or to a defined population of them, so homogeneous cell populations are not available in sufficient quantities to characterize brain neurons using biochemical approaches. Also, lineage relationships of neurons with their immediate precursors have remained unclear.

With regard to the use of ES cell-derived neurons for transplantation, it is desirable to obtain defined progenitor cells giving rise to known progeny, as opposed to a mixture of cells including some that may continue to divide and form tumours (ref. 3, 4). Heterologous cells may also interfere with trophic and/or guidance signals from the host tissue which promote integration of the implanted tissue into the brain. The cell type implanted is functionally important, for example dopaminergic neurons in particular may be required to treat diseases such as Parkinson's disease, so increased control over the precursor and neuronal cell sub-types generated is desirable for such medical applications. Reduction of cellular heterogeneity is needed to reduce undesirable side effects, lower the risk of tumours, and to improve the therapeutic potential by increasing the proportion of cells having the desired neuronal lineage.

Recently, progress has been made through use of inductive signals and of transcription factors to substantially enrich for subtypes of neurons, including in particular dopaminergic neurons and motor neurons. Thus, transcription factors like Nurr1 (ref. 5) or co-culture of stem cells with other types (ref. 6) markedly increase the generation of dopaminergic neurons, while the addition of extrinsic factors including sonic hedgehog increases that of motor neurons, which were also shown to integrate into host tissue after transplantation (ref. 7). But in spite of this progress, still very little is known about the in vitro generation of defined neuronal precursors that may give rise to specified neuronal phenotypes.

A number of different protocols exist describing the generation of neuronal and glial cells (refs 14, 15, 25-31). As realized early on with the embryonic carcinoma cell line P19, treatment of pluripotent cell aggregates with retinoic acid triggers neuronal differentiation (ref. 32). Subsequently, it has been observed that treatment of ES cells-derived embryoid bodies (EBs) with RA also promotes neural and represses mesodermal gene expression (ref. 33). EBs are three-dimensional aggregates arising by aggregation and proliferation of ES cells. EBs may be produced by culturing ES cells on a substrate to which they cannot adhere, typically a bacterial culture dish (see for example ref. 41).

One method for generating neural cells from ES cells, as exemplified in Bain et al. (ref. 14) and Li et al. (ref. 10) includes the steps of:
culturing ES cells;
forming EBs;
contacting the EBs with retinoic acid (RA);
dissociating the EBs; and
plating and culturing the dissociated EB cells.

Usually, initial ES cell culture is done on a feeder cell layer support (inactivated fibroblasts) to keep the ES cells in a colony form of pluripotent undifferentiated ES cells. Fibroblasts are believed to support the undifferentiated state of ES cells. Leukaemia inhibitory factor (LIF) may be included in the culture medium to inhibit differentiation. However, it has been observed that even in the presence of LIF, some ES cells have a tendency to differentiate and that during EB formation, cells of different lineages can be observed (ref. 3, 34).

In the methods described in Bain et al. and Li et al., (ref. 10, 14, 15), cultured ES cells were treated with trypsin and/or triturated into small clumps, which were then seeded in non-adherent cell culture for EB formation. The cells were cultured for four days without RA, then for four days with RA in the medium, after which the EBs were dissociated and plated on laminin-coated dishes. The plated cells were cultured in serum-containing media.

Using this method, Bain et al. report the production of a culture consisting of a population of flat cells adhering tightly to the laminin-coated substrate and a population of neuron-like cells mostly lying on top of the flat cells. Around 38% of the cells were observed to have a neuronal morphology after two days' culture. These cultures were of mixed composition consisting of various types of neurons, especially GABAergic neurons.

Some approaches have made use of selection markers of neuronal precursors, thereby eliminating cells other than neuronal precursors during the differentiation procedure. Neural progenitors generated from ES cells have been defined mostly by the expression of intermediate filament markers such as nestin (ref. 9) or by transcription factors such as the sox genes (ref. 10). Li et al. used lineage selection to enrich their heterogeneic cell populations for Sox2 expressing cells by eliminating Sox2-negative cells (ref. 10). While selection methods have proved useful to enrich for neuronal precursors, it is doubtful whether the selected precursors can be used to generate defined neuronal phenotypes. The available data in Li et al. indicate that Sox-positive cells may give rise to most cell types found in the central nervous system (CNS) as opposed to a defined sub-lineage. Thus, while selection of Sox-positive cells may increase the proportion of neuronal precursor cells in an ES-cell derived population, it appears unlikely that such selection could be used to enrich specifically for a single sub-type of neuronal precursors or neurons.

Other methods have been established without using RA. For example methods used in Okabe et al. (ref. 27, ref. 43) did not use RA, but included an intermediate step of culturing the formed EBs on an adherent substrate in a special medium before dissociation. An intermediate step is also used in Abe et al. (ref. 30), in which cultured EBs are transferred to a substrate onto which they can adhere. They are then cultured in the adhered state prior to dissociation with trypsin.

Some methods, such as those of Abe et al. (ref. 30) and Okabe et al. (ref. 27) have included the use of basic fibroblast growth factor. Abe et al subsequently used mitotic inhibitors, which caused the death of neurons and of non-neuronal cells (ref. 30).

### THE PRESENT INVENTION

We have discovered means by which differentiation of ES cells to neural cells can be optimised to produce surprising advantages in terms of generation of defined neural cell lineages and homogeneity of neural cell populations. Accordingly, the present invention provides improved methods of inducing and/or promoting development and/or differentiation of ES cells into neurons or neuronal precursor or progenitor cells, to generate neural cells from ES cells in vitro.

In preferred embodiments, methods of the invention allow the production of a substantially homogeneous neural cell population wherein the neural cells are substantially all of a single defined neuronal lineage, phenotype, cell type and/or are at the same stage of differentiation.

As described in detail elsewhere herein, we devised procedures to obtain homogeneous neuronal precursors, which were identified as radial glial cells. During subsequent culture, the ES cell-derived radial glial cells progressively differentiated into pyramidal neurons. The precursors and neurons generated by methods of the invention were substantially homogeneous, showing higher % yield of neuronal cells of a single lineage compared with methods of the prior art.

Thus, in more preferred embodiments, methods of the invention can produce substantially pure populations of radial glial cells and of pyramidal neurons, one of the most important neuronal populations of the cortex that has been difficult to generate in the prior art using ES cells.

Given the high level of homogeneity of the neural precursor/progenitor cells produced by the present invention, these cells are suitable for further differentiation and/or maturation to produce neuronal cells of a defined lineage. Precursor/progenitor cells may be differentiated to produce pyramidal neurons as shown herein, or may be manipulated by extrinsic or intrinsic factors to generate other neuronal populations.

The advantage in cell numbers and homogeneity provided by the present invention contrasts with cells produced by prior art methods of neurogenesis and neural cell differentiation, and with the limited numbers of primary neurons that may be prepared from mice or rat brains.

Biochemical studies were previously hampered by the limited numbers of neural cells that could conveniently be produced by prior art methods. The present invention facilitates the study of biochemical and genetic mechanisms involved in neural cell development, especially in the transition from neural precursors to neuronal cells. ES cells can be easily genetically manipulated and produced in unlimited numbers, and the present invention is ideally suited for the production of large numbers of neurons of a defined lineage for biochemical study.

In addition, as ES cells can readily be genetically manipulated or isolated from mice carrying relevant mutations, the present invention facilitates comparison of wild-type and mutant neurons and the identification of mechanisms causing the loss of specific cell types in neurodegenerative diseases. While genetic manipulation of ES cells is easy, manipulation of primary neurons is extremely difficult, especially stable manipulation. Genetic manipulation of ES cells can provide a homogeneous modified line in which the whole progeny contains the same mutation and can be achieved in one or two months, whereas establishing a mouse line with a stable mutation can take years. Thus, by providing methods of producing precursor, progenitor and neuronal cells in vitro from ES cells, the present invention avoids the need to establish transgenic mouse lines and thereby allows study of mutant neurons on a level that was previously not practical.

Methods of the invention also provide a cellular assay system for neurons (e.g. neurite elongation, neuronal cell death, neurogenesis and synaptogenesis). Such assays are needed in the field, but their use and performance have been limited because neurons could not be conveniently produced in sufficient quantities. The present invention enables neurons to be produced in greater quantities and with far greater homogeneity than before, thus allowing performance of neuronal assays.

Neurons and/or neuronal precursor/progenitor cells produced by the invention are also suitable for medical applications such as implantation into the brain to treat neurodegenerative disease or neuronal loss. Owing to the greater homogeneity of neural cells of the desired sub-type as produced by the present invention, the therapeutic potential of the treatment is improved and the risk of tumours following implantation reduced.

### DETAILED DESCRIPTION OF THE INVENTION

The present invention relates to methods of producing or generating neural cells e.g. neurons and/or neuronal precursor/progenitor cells, promoting or inducing differentiation of ES cells into neuronal precursor or progenitor cells, and to methods of promoting or inducing differentiation or maturation of the precursor or progenitor cells into neurons.

The present invention relates to an improved in vitro method of inducing and/or promoting development and/or differentiation of embryonic stem (ES) cells into neuronal precursor or progenitor cells or neurons, and/or producing or generating neural cells, the method comprising
culturing ES cells;
forming embryoid bodies (EBs);
contacting the EBs with retinoic acid (RA); and
dissociating the EBs;
   in combination with one or more further features or steps described hereinafter.

In methods of the invention, the dissociated EB cells are neuronal precursor cells or neuronal progenitor cells. Dissociation of EBs can thus produce a culture of neuronal precursor or progenitor cells.

Optionally, the method further comprises
plating the dissociated EB cells, thereby obtaining a plated culture of neuronal precursor or progenitor cells.

The method may comprise culturing the neuronal precursor or progenitor cells to produce neurons. Thus, in some embodiments methods of the invention comprise plating and culturing the dissociated EB cells to produce neurons.

Methods of the present invention further comprise one or more features/steps as described below. Any feature or step may be used alone or used in combination with any other feature or step, unless otherwise indicated by context.

### Feeder-Free ES Cell Culture

Preferably, the method comprises culturing ES cells in the absence of feeder cells (typically inactivated fibroblasts).

Methods may include initial culture of ES cells with feeder cells, followed by culture without feeder cells. Feeder cells may be diluted out and removed by repeated passage of the ES cells. It is preferred that at least one, more preferably at least two passages without feeder cells are performed before embryoid body formation. Thus, feeder cells are preferably absent from the ES cell culture used for EB formation. “Passage” comprises dissociating cells, and re-plating a number of cells. For example, passage may comprise detaching/dissociating the cells from the culture dish (normally using trypsin), dissociating aggregates of cells and re-plating a number of dissociated ES cells (adherent culture) and culturing the ES cells.

Appropriate culture media are described elsewhere herein. Leukaemia inhibitory factor (LIF) may optionally be included in the ES cell culture medium.

### Selection and Plating of ES Cells for EB Formation

We have recognised that the proliferative status of ES cells affects their pluripotency, and that the density of cells plated in the method has an impact on their ability and tendency to differentiate. We have found that by selecting and plating proliferating ES cells at a controlled cell density, greater numbers of neuronal precursors having a defined cell lineage may be obtained and fewer heterogeneous cells produced.

Preferably, methods of the invention comprise selecting highly proliferative and/or morphologically homogeneous ES cells for EB formation. Preferably, methods comprise plating a measured/estimated/defined/determined number or density of said ES cells for EB formation. Preferably, the method comprises selecting a measured, estimated, defined or determined number of ES cells for plating to produce EBs.

The method preferably comprises measuring, estimating, observing or determining:

the proliferation state of the ES cells (which may be measured or estimated by determining the doubling time, increase in cell number, or any other appropriate measure);
the morphology of the ES cells; and/or
the number or density of ES cells plated for EB formation.

Thus, preferably the cells are plated at a measured, estimated or determined density. Measurement, estimation or determination of cell number may be by any method known in the art, e.g. comprising counting the cells in a given area under a microscope, or using conventional cell counters as Casy®1 (Scharfe System GmbH). Cell morphology may be observed by microscopic observation.

Each of these points is discussed in more detail below.

### Production and Selection of Highly Proliferative Cells

Highly proliferative cells may be cells produced by a particular method of culturing as described herein. We have found that the proliferation state of ES cells can be varied through the method of culturing the ES cells.

ES cell culture or passage preferably produces highly proliferative cells. Preferably passage is repeated about every 2 days, and ES cell culture preferably comprises at least two passages on feeder cells followed by at least two passages without feeder cells. ES cells should be deprived from feeders in a highly proliferative state, for example by splitting a 10 cm dish of ES cells on feeder cells and re-plating (e.g. taking ¼ by volume of the cell suspension and re-plating in the original volume of medium) without feeders should give a 60% confluent ES cell culture already again the next day. Passage without feeders may comprise plating about 0.5×10⁵ cells per cm².

Preferably, culturing ES cells comprises measuring, estimating or determining the number or density of cells plated for ES cell culture.

Highly proliferative ES cells may be ES cells produced by culturing or passaging ES cells substantially as follows (normally without feeder cells):
plating ES cells at a density of between about 0.3×10⁵ and 4×10⁵ cells per cm², e.g. between about 0.5-2×10⁵, and preferably about 1×10⁵ cells per cm²; and
recovering/dissociating the ES cells 2 days after plating, and optionally re-plating.

The ES cells should be recovered by splitting (dissociating) 2 days after plating. Normally, this culture procedure (passage) should be performed at least two or three times, before selecting highly proliferative cells for EB formation.

For example, about 2×10⁶ cells may be plated in a 10 cm² cell culture dish. The above procedure normally allows between 10×10⁶ and 35×10⁶ cells per 10 cm² to be recovered after 2 days, e.g. between 10-20×10⁶.

Proliferation state may be measured in terms of doubling time of the ES cells. Methods of the invention may comprise measuring doubling time of the ES cells, and selecting highly proliferative cells. For example, highly proliferative cells may have a doubling time of 8 hours or less, 16 hours or less, or 24 hours or less, normally between 8 and 24 hours.

### Morphological Characteristics

ES cells used for EB formation are preferably morphologically homogeneous, wherein all or substantially all the ES cells have the same or similar morphological features.

Preferably, methods of the invention comprise selecting morphologically homogeneous ES cells for EB formation, and plating those cells for EB formation. Preferably, all or substantially all (e.g. at least 80%, at least 90%, at least 95%, at least 98% or at least 99%) the ES cells selected for EB formation have one or more, and most preferably all, of the following morphological features (in culture without feeder cells): growth in a flat monolayer; neighbouring cells not in direct contact with one another (but nevertheless densely packed); large nuclei; many nucleoli; cells not growing on top of one another or in colony-like form. Preferably, the cells are densely packed, e.g. the cells are at a density of about 20×10⁶ cells per 10 cm² dish (2×10⁵ cells per cm²), preferably at a density of between about 10-30×10⁶ cells, e.g. 15-25×10⁶ cells, per 10 cm² dish. The method may comprise observing one or more preferred morphological features of the ES cells, and/or selecting cells having one or more of these features.

Cell morphology may also be used as an indicator of proliferation state. Highly proliferative cells preferably have one or more, and preferably all, of the above-listed morphological features.

Preferably, all cultured ES cells are derived from a single ES cell, e.g. an earlier step of the method may comprise selecting a single ES cell colony and culturing ES cells from that colony. Uniformity and homogeneity, including morphological homogeneity, of ES cells in the culture can thereby be increased.

### Plating Density

For EB formation, the thus generated highly proliferative cells and/or morphologically homogeneous cells should normally be plated using between around 0.5×10⁶ and 5×10⁶ cells per 15 ml culture medium for EB formation, preferably 2.5-2.5×10⁶ cells, e.g. 3×10⁶ cells in 15 ml medium. Between about 0.3-3.5×10⁵ cells.ml⁻¹ should normally be plated, preferably 1.6-2.5×10⁵ cells.ml⁻¹, most preferably 2×10⁵ cells.ml⁻¹. 15 ml medium is a preferred volume, although 10 ml, or between 10-15 ml, can be used, normally on 10 cm plates.

The density of cells plated for EB formation should be adjusted according to the proliferation state of the ES cells used. Thus, if the ES cell culture is more dense, then more cells should be plated, whereas if the culture is less dense, then fewer cells should be plated. We have found that best results are obtained using most rapidly proliferating ES cells.

As an example, ES cells of homogeneous morphology having a doubling time of between about 12-16 hours may be selected and plated at a density of about 0.5×10⁵ cells per cm².

### Dissociation of Cells

In methods of the invention generally, dissociation of cells preferably comprises dissociating the cells (ES cells or EBs) to form a suspension of single cells substantially lacking aggregates of more than 2 or 3 cells. Preferably, the suspension is of entirely singly dissociated cells (i.e. the suspension has no aggregates of cells). Preferably, over 90%, 95%, 98% or 99% of cells in the suspension are singly dissociated. Preferably, less than 5% of cells in the suspension form aggregates of 4 or more cells.

Trypsin (e.g. 0.05%) and/or trituration may be used to dissociate the cells, using methods described in detail elsewhere herein.

ES cells should be well dissociated prior to plating for EB formation. Thus, preferably methods of the invention comprise dissociating ES cells to form a suspension of single cells substantially lacking aggregates of more than 2 or 3 cells. Preferably, the suspension is of entirely singly dissociated cells (i.e. the suspension has no aggregates of cells). Preferably, over 90%, 95%, 98% or 99% of cells in the suspension are singly dissociated. Preferably, less than 5% of cells in the suspension form aggregates of 4 or more cells.

Methods of the invention may comprise determining or estimating the level of dissociation of the ES cells. Preferably, methods comprise dissociating ES cells and selecting a suspension of dissociated cells according to the invention. Microscopic observation or conventional cell counters may be used to determine or estimate the extent of dissociation. For example, using the Casy®1 cell counter, cell peaks at higher diameter are detected if aggregates are present.

### Direct Dissociation of EB Cells

EBs are cultured in suspension culture and then the EB cells are dissociated, producing a suspension of dissociated EB cells. Normally, the EBs are dissociated after 8 days, i.e. on the 8th day following plating of cells for EB formation or four days after addition of RA. Dissociation may be performed earlier or later than this, but is normally between 3 and 5 days after addition of RA. The person skilled in the art is able to determine experimentally the optimum time for dissociation.

Preferably, the EBs are not plated on adherent substrate prior to dissociation, but instead maintained in non-adherent culture until dissociation of the cells. Thus, the EBs should preferably be dissociated prior to plating and not plated directly.

Dissociation of EBs normally comprises incubating the EBs with trypsin (normally 0.05%, or between 0.01-0.5%). Preferably, methods of the invention comprise filtering the suspension of dissociated EBs to remove cell clumps, e.g. the cells may be filtered through a mesh or strainer, typically a nylon mesh or strainer. Normally a 40 μm cell mesh or strainer is used. In embodiments of the invention, the pore or mesh diameter is preferably at least 20, 30 or 40 μm, and preferably 100, 80, 60 or 50 μm or less.

### Storage of EB Cells

Methods of the invention may comprise storing dissociated EB cells e.g. freezing the cells in liquid nitrogen. For example, storing may comprise centrifuging the cells, resuspending the cells after centrifugation in EB medium+10% DMSO, and freezing the cells in liquid nitrogen. Thus, in some embodiments the method comprises dissociating the EBs, and storing the dissociated EB cells. A convenient, ready supply of neural precursors may thus be obtained.

Frozen stocks may be thawed as and when needed, e.g. for plating and culture to produce neurons. The possibility of storing such precursors for later use has not previously been published in the field. Normally the cells are thawed and immediately after thawing are resuspended in medium, typically 10 ml N2 medium, centrifuged (typically for 5 min at 1000 rpm room temperature) and resuspended (typically in N2 medium).

### Plating Density of Dissociated EB Cells

In aspects where the EB cells are plated, we have found that plating density of EB cells is important for cell survival and differentiation. Plating too thinly reduces cell survival, while plating too densely adversely affects the speed of differentiation. Density of plating also affects purity of culture, i.e. amount of non-neuronal versus neuronal cells. Preferably, between about 0.5×10⁵ and 2.5×10⁵ dissociated EB cells per cm² should be plated, e.g. between about 1-2×10⁵, most preferably about 1 to 1.5×10⁵ cells per cm².

Methods of the invention may comprise measuring, estimating or determining the number or density of EB cells plated, using methods described elsewhere herein.

### Change of Culture Medium

We have observed that, remarkably, a great increase in cell survival is achieved if culture medium is changed about 2 hours after plating dissociated EB cells. This finding opens the possibility of producing long-term neuronal cultures, which until now have been uncommon in the field.

In this context, changing the culture medium means refreshing or replacing the culture medium. The new medium is preferably of the same composition as the medium in which the dissociated EB cells were originally or previously plated, i.e. the same type of medium is used. Medium of similar composition might be used, but preferably the composition is the same as that previously used. For example, the medium may be N2 medium.

Accordingly, methods of the invention preferably comprise changing the culture medium following dissociation of EBs and plating of the dissociated EB cells in culture medium. Preferably, the culture medium is changed between about 1 and 6 hours after plating.

The culture medium may be changed within 6 hours of plating, preferably within 5, 4, 3 or 2.5 hours of plating. The culture medium may be changed after at least about 1 hour, 1.5 hours or 2 hours after plating.

Most preferably, the culture medium is changed between about 1 and 3 hours after plating, more preferably between about 1.5 and 2.5 hours, and most preferably about 2 hours.

### Culture of Plated Dissociated EB Cells

Dissociated EB cells are preferably plated in N2 medium.

After two days, the medium is preferably changed to a suitable medium for neuronal differentiation, such as the “complete medium” (see Examples). The choice and composition of medium may depend on the desired neuronal lineage. For example, the complete medium used herein was based on Brewer's medium and designed to promote development of pyramidal neurons. Other media or factors may be chosen to support a different neuronal lineage, for example Shh (Sonic hedgehog) to produce cholinergic motoneurons.

We found that precursors produced according to the present invention were able to differentiate into a number of different specific neuronal lineages, including motoneurons, following implantation into chick embryos.

In some embodiments, it is preferred that the culture medium does not contain T3. The complete medium used herein was based on Brewer's medium, but T3 was omitted from the composition. It is possible that T3, which is found in FCS, may inhibit neuronal differentiation.

Preferably, Neurobasal medium is not used. Neurobasal medium+B27 supplement (both available from GIBCO) are typically used in the prior art for neuronal culture. However, we have observed that Neurobasal medium may promote glial cell development rather than neuronal cell development. Thus, use of Neurobasal medium may lead to the undesirable presence of glial cells among the neuronal cells produced. In contrast, the complete medium used herein appears to suppress glial cell development in favour of neuronal development.

Preferably, the plated cells (dissociated EB cells, neuronal precursor/progenitor cells) are cultured in the absence of serum, not cultured in the presence of serum. (Serum may be used to inactivate trypsin after cell dissociation, but should then be removed, e.g. by centrifugation to pellet the cells and substantially complete removal of supernatant.)

Preferably, growth factors (especially EGF, FGF/bFGF and PDGF) are absent from the culture media and the precursor or progenitor cells are not cultured in the presence of these or other growth factors.

Methods may comprise culturing neurons, and the neurons are also preferably not cultured in the presence of serum and preferably not cultured in the presence of growth factors, especially EGF, FGF/bFGF or PDGF.

Furthermore, methods of the invention do not require and preferably do not include positive or negative selection steps e.g. Sox-2 genetic selection, to enrich for neural cells or neurons, although if desired such selection procedures may be used. Methods of the present invention produce substantially homogeneous neural cell populations even without a selection step. Preferably, methods of the present invention do not include a step of negative selection against non-neural or non-neuronal cell types (e.g. dividing cells). Preferably, methods of the invention do not include a step of positive selection, to enrich for neural cells or neurons. Known selection methods include genetic selection e.g. Sox-2 selection against Sox-2 negative cells, and contacting cells with a negative selection agent to inhibit and/or kill non-neural or non-neuronal cells, e.g. contacting cells with an anti-mitogen such as AraC or FRDU to inhibit and/or kill dividing cells.

### ES Cells

Embryonic stem cells are pluripotent stem cells isolated from the inner cell mass of the mammalian blastocyst. The embryonic stem cells used in the invention may be from any mammal, which may be human or non-human, such as guinea pig, rat, mouse or other rodent, cat, dog, pig, sheep, goat, cattle, horse or primate e.g. monkey. Typically mouse ES cells are used.

In the present invention ES cells are normally pluripotent cells, not totipotent cells, and not able to produce germ cells. The ES cells used in the examples herein are pluripotent. Optionally, totipotent ES cells may be used.

A number of ES cell lines are known in the art and may be used in the present invention (e.g. J1, E14).

ES cells designed to allow selection procedures may be used, e.g. Sox2 selection.

As described elsewhere herein, ES cells used in the present invention may be targeted cell lines or genetically manipulated lines containing an introduced gene or a mutated gene or overexpressing an endogenous gene.

ES cell lines comprising a reporter gene operably linked to a promoter (e.g. a promoter for neuron-specific expression) may be used. We describe use of a Tau-GFP line herein. Properties of the Tau locus include high relevant expression levels of inserted cDNAs, high recombination efficiency, expression only in neurons, and Tau knockouts have no apparent phenotype. We used the tau locus to insert cDNA to be investigated. Tau can be easily replaced by various cDNAs, or cDNAs may be inserted at the Tau locus (such that their expression is operably linked to the Tau promoter), to rapidly establish high level of stable expression specifically in neurons (ref. 42).

### Neural Cells

As used herein, a neural cell is a cell of the nervous system, and includes a neural stem cell, neuronal precursor or progenitor cell, and a neuron (neuronal cell), unless otherwise indicated by the context. The terms “neuron” and “neuronal cell” are used interchangeably.

By “stem cell” is meant any cell type that can self renew and, if it is a multipotent or neural stem cell, can give rise to all cell types in the nervous system, including neurons, astrocytes and oligodendrocytes. A stem cell may express one or more of the following markers: Oct-4; Sox1-3; stage specific embryonic antigens (SSEA-1, -3, and -4) (Tropepe. et al., 2001, Neuron 30, 65-78). A neural stem cell may express one or more of the following markers: Nestin; the p75 neurotrophin receptor; Notch1, SSEA-1 (Capela and Temple, 2002, Neuron 35, 865-875).

By “neural progenitor cell” is meant a daughter or descendant of a neural stem cell, with a more differentiated phenotype and/or a more reduced differentiation potential compared to the stem cell. By precursor cell it is meant any other cell being or not being in a direct lineage relation with neurons during development but that under defined environmental conditions can be induced to transdifferentiate or redifferentiate or acquire a neuronal phenotype.

By lineage” is meant the progeny of, or cells derived from, one defined cell type. By “sub-lineage” is meant a subtype of a certain lineage.

### Detection of Markers and Identification of Cell Types

Methods of the invention preferably produce a population of cells in which at least 80%, at least 85%, at least 90% or at least 95% of cells are neuronal precursor/progenitor cells e.g. radial glial cells, or neurons e.g. pyramidal neurons. Methods preferably comprise identifying at least 80%, at least 85%, at least 90%, at least 95%, at least 98% or at least 99% of cells as neuronal precursor/progenitor cells e.g. radial glial cells, or neurons e.g. pyramidal neurons. Neuronal cell culture methods of the invention preferably produce a population of cells having fewer than 5% astrocytes, e.g. fewer than 4%, 3%, 2% or 1%.

Methods of the present invention as described above are preferably such as to achieve these proportions. The present invention provides methods of achieving, producing or generating these proportions of cells using one or more method steps and features as described above.

Methods of the invention may comprise identifying dissociated EB cells as neuronal precursors, or (following plated culture) as neurons. The method may comprise determining, observing or confirming that at least 80%, at least 85%, at least 90% or at least 95% of cells, and identifying at least 80%, at least 85%, at least 90%, at least 95%, at least 98% or at least 99% of cells are neuronal precursor/progenitor cells e.g. radial glial cells, or neurons e.g. pyramidal neurons. Typically, fewer than 5% cells produced through neuronal cell culture methods described herein are astrocytes, e.g. fewer than 4%, 3%, 2% or 1%.

Cell lineage and/or cell-type may be determined by observing cell morphology e.g by microscopic inspection. The method may comprise observing neuronal precursor/progenitor morphology or neuronal cell morphology, in at least these proportions of cells generated. Neuronal precursors/progenitors may be elongated and/or have a bipolar spindle-morphology. Neuronal lineage may be determined by observing neuronal morphology, e.g. pyramidal neurons are of triangular shape and have branching neuritic extensions, while cholinergic neurons have a bipolar morphology.

Cells generated according to methods of the invention may alternatively or additionally be identified through detection of markers, typically cell-surface markers recognised by antibodies. The method may comprise detecting the presence of one or more markers, whose presence indicates that the cell is a particular lineage or sublineage, or a particular cell type or sub-type. The skilled person knows markers that may be identified and used as an indication of lineage or cell type.

For example, the method may comprise detecting the presence of the marker Pax6 on the cells and identifying the cells as neuronal precursors, e.g. radial glial cells. Other markers that may be detected include Nestin, RC2 and BLBP, which are present on radial glial cells, and p75, GluR1, synaptophysin, Trks (e.g. TrkA, TrkB, TrkC) and APP, which are present on certain neuronal cells.

The method may comprise detecting a high percentage of cells expressing neuronal precursor markers, e.g. at least 80%, at least 85%, at least 90% or at least 95% of cells, and identifying at least 80%, at least 85%, at least 90%, at least 95%, at least 98% or at least 99% of cells as neuronal precursors.

The method may comprise detecting a high percentage of cells expressing neuronal cell markers, e.g. at least 80%, at least 85%, at least 90% or at least 95% of cells, and identifying at least 80%, at least 85%, at least 90%, at least 95%, at least 98% or at least 99% of cells as neurons, preferably neurons of a defined lineage, e.g. pyramidal neurons or dopaminergic neurons.

Thus, the method may produce substantially homogeneous populations of neuronal precursor cells or neurons. At least 80%, at least 85%, at least 90%, at least 95%, at least 98% or at least 99% of cells may be of the same type/lineage or sub-type/lineage, e.g. neuronal precursors of the same type such as radial glial cells or neurons of the same lineage such as pyramidal neurons.

The examples herein provide details of the time course of expression of various markers and morphological development over time. Methods of the invention may comprise detecting markers and/or observing particular morphology at certain times after EB dissociation (as noted in the examples), e.g. observing neuronal morphology less than 2 days after EB dissociation and/or detecting expression of Trk receptors after about 7 days. For example, it is demonstrated herein that about 99% of cells produced by a method of the invention were radial glial cells, as indicated by detection of RC2+ expression by 99% of dissociated EB cells. It is also shown herein that at least 80% neurons may routinely be produced by methods of the invention, as indicated by measuring expression of or counting vGLUT1 and GFP after about 7 days after EB dissociation.

Percentages may be calculated as % viable cells or % cells expressing a nuclear marker e.g. DAPI or Hoechst.

Formation of EBs and Treatment with RA

In methods of the invention generally, EBs are formed and cultured in culture medium. During EB formation and culture, culture medium is typically changed every two days.

Normally in methods of the invention EBs are cultured in the presence of RA for one or more days, typically for two, three, or preferably four days, or up to five, six, seven or eight days. The EBs may be cultured initially in the absence of RA for one or more days, normally between two and six days, typically for two, three, or preferably four days, or up to five or six days prior to contact with RA. A 4-day/4+day procedure was used by Bain et al. and by Li et al.

The skilled person can select an appropriate concentration of RA. For example, the concentration may be e.g. at least 0.25 μM, at least 0.5 μM or at least 1 μM. The concentration may be e.g. 10 μM or less, 7.5 μM or less or 5 μM or less. Preferably, the concentration is between 0.5 and 5 μM inclusive. For example, the concentration may be 1 μM or 5 μM.

### Neural Cellular Assays

Further aspects of the present invention provide cellular assay methods performed with neuronal precursor or progenitor cells or neuronal cells, which are normally in vitro-generated cells (not primary neurons) and preferably are cells produced by a method of the invention. The assay methods may include a method of the invention as described herein for producing neuronal precursor or progenitor cells or neurons. Methods of the invention may comprise performing a method of the invention as described herein for neural differentiation (producing neuronal precursor or progenitor cells or neurons), and further comprise the steps of a cellular assay method described here.

Thus, neural differentiation methods described above may be used in the context of assays.

Furthermore, as we have provided substantially homogeneous cultures/populations of neuronal precursor or progenitor or neuronal cells for the first time, the invention further provides assay methods performed with substantially homogeneous cultures/populations of neuronal precursor or progenitor or neuronal cells, which may or may not be produced by neural differentiation methods of the present invention, but are normally produced by in vitro methods.

Assay methods of the invention may comprise detecting, quantifying, observing or determining one or more characteristics of neuronal precursor or progenitor cells, or neurons (“neuronal characteristics”), e.g. neuritic growth or neurite elongation/degeneration, neuronal shape, neuronal cell death, neurogenesis, neuronal differentiation, electrical activity, synaptogenesis and/or neuronal cell markers.

In some embodiments, assay methods of the present invention may comprise a neural differentiation method described herein for producing neuronal precursor or progenitor cells or neuronal cells, wherein the method further includes culturing the ES cells and/or EBs under a test condition; and detecting, quantifying, observing or determining one or more neuronal characteristics of the neuronal precursor or progenitor cells or neuronal cells.

In other embodiments, assay methods of the present invention may comprise culturing neuronal precursor or progenitor or neuronal cells under a test condition; and detecting, quantifying, observing or determining one or more neuronal characteristics of the cells. Optionally, the cells may be produced and/or cultured according to neural differentiation methods described elsewhere herein.

Assay methods may optionally comprise comparing neuronal characteristics under the test condition (“test culture”) with neuronal characteristics of cells cultured under a second condition (“control culture”), optionally with historical data from cells cultured under a second condition. Methods may comprise culturing cells under the second condition.

Thus, assay methods may comprise a neural differentiation method described herein for producing neuronal precursor or progenitor cells or neuronal cells, including culturing ES cells or EBs under a first and a second condition; and comparing one or more neuronal characteristics of neuronal precursor or progenitor cells or neuronal cells cultured under the first condition with the same neuronal characteristic or characteristics in neuronal precursor or progenitor cells or neuronal cells cultured under the second condition, respectively.

Culturing under the test condition or first condition may comprise contacting the cells with a test compound or exposing the cells to a test compound or culturing the cells in the presence of a test compound, which may be added to or included in culture medium. Culturing under the second condition may comprise culturing the cells in the absence of the test compound, or not contacting the cells with or exposing the cells to the test compound.

The test compound may be any molecule and may be from a library of test compounds. In some embodiments, the test compound is a double-stranded RNA (dsRNA) molecule and culturing under the first or test condition comprises exposing ES cells or EB cells to the double-stranded RNA molecule and thereby inhibiting a gene in the cells through RNA interference (RNAi).

dsRNA has been found to be even more effective in gene silencing than both sense or antisense strands alone (Fire A. et al Nature, Vol 391, (1998)). dsRNA mediated silencing is gene specific and is often termed RNA interference (RNAi) (See also Fire (1999) Trends Genet. 15: 358-363, Sharp (2001) Genes Dev. 15: 485-490, Hammond et al. (2001)Nature Rev. Genes 2: 1110-1119 and Tusch1 (2001) Chem. Biochem. 2: 239-245).

RNA interference is a two step process. First, dsRNA is cleaved within the cell to yield short interfering RNAs (siRNAs) of about 21-23 nt length with 5′ terminal phosphate and 3′ short overhangs (˜2 nt) The siRNAs target the corresponding mRNA sequence specifically for destruction (Zamore P. D. Nature Structural Biology, 8, 9, 746-750, (2001)

RNAi may be efficiently induced using chemically synthesized siRNA duplexes of the same structure with 3′-overhang ends (Zamore P D et al Cell, 101, 25-33, (2000)). Synthetic siRNA duplexes have been shown to specifically suppress expression of endogenous and heterologeous genes in a wide range of mammalian cell lines (Elbashir S M. et al. Nature, 411, 494-498, (2001)). siRNA duplexes containing between 20 and 25 bps, more preferably between 21 and 23 bps, of the sequence to be inhibited may be used.

Alternatively siRNA may be produced from a vector, in vitro (for recovery and use) or in vivo.

In other embodiments, the test compound may be nucleic acid (DNA, cDNA or RNA), optionally encoding a gene e.g. cDNA. Thus, the test compound may be a vector encoding a gene, wherein exposing cells to the nucleic acid or vector results in the gene being expressed in the cells. In one embodiment, the vector may comprise a nucleic acid sequence according to the invention in both the sense and antisense orientation, such that when expressed as RNA the sense and antisense sections will associate to form a double stranded RNA. This may for example be a long double stranded RNA (e.g., more than 23 nts) which may be processed in the cell to produce siRNAs for RNAi (see for example Myers (2003) Nature Biotechnology 21:324-328).

In other embodiments, the test compound may be an antibody.

Assay methods may thus identify a compound or condition that increases or reduces the characteristic of interest.

Comparisons are typically performed with neuronal cells, e.g. one week after plating dissociated EB cells.

The test and control cultures are typically two separate cultures, cultured under otherwise identical conditions. Where the condition is the presence of a test compound, especially where it is nucleic acid, culturing under the test or first condition may comprise exposing cells (typically ES cells or dissociated EB cells) to the test compound and then culturing the cells.

Neuronal characteristics (e.g. neuritic growth or neurite elongation) may be detected by causing or allowing expression of a neuron-specific reporter gene, and detecting or quantifying expression of the reporter gene. The reporter gene may encode a fluorescent protein e.g. green fluorescent protein (GFP). A reporter gene may be targeted to or operably linked to a neuron-specific locus or promoter such as the tau locus or promoter for neuron-specific expression. Neuron-specific reporter gene expression from the tau locus has been described (Tucker et al. (42)). Expression of the reporter gene is switched on as soon as the cell differentiates into a neuron, and only in neurons, not in precursors or other cell types in the nervous system. Methods of the invention including neuronal cell assays may use a cell line (ES cells) containing a reporter gene having neuron-specific expression, the reporter gene being operably linked to a promoter or locus expressed only in neurons (e.g. the Tau-GFP line as described elsewhere herein).

The invention also provides assay methods of identifying an agent that inhibits or reduces an increase in a neuronal characteristic produced by a condition known to increase that characteristic or associated with an increase in the characteristic (e.g. in some embodiments, wherein the condition is culturing in the presence of amyloid beta peptide), i.e. identifying an agent that reduces or inhibits the effects associated with such a condition.

Such an assay may comprise:
culturing neuronal precursor or progenitor or neuronal cells in the presence of a test agent and under a condition known to increase or associated with an increase in the neuronal characteristic;
culturing neuronal precursor or progenitor or neuronal cells in the absence of the test agent and under a condition known to increase the neuronal characteristic;
quantifying or determining levels of the neuronal characteristic; and
comparing levels of the neuronal characteristic in the presence of the test agent with levels of the neuronal characteristic in the absence of the test agent;
wherein a lower level of the neuronal characteristic in the presence of the test agent compared with the absence of the test agent indicates that the agent inhibits or reduces an increase in the neuronal characteristic produced by or associated with the condition.

A condition known to increase or associated with an increase in the neuronal characteristic may be a condition identified by an assay method of the invention as being a condition that increases the neuronal characteristic.

For example, where the test compound is nucleic acid (e.g. dsRNA), culturing under a condition known to increase the neuronal characteristic may comprise exposing cells to the nucleic acid and then culturing the cells.

Culturing with the test agent and culturing under the condition may be performed simultaneously, or culturing with the test agent may be performed before culturing under the condition, or culturing under the condition may be performed before culturing with the test agent. The skilled person can determine an appropriate order, and in some embodiments one order may be preferred over another order. For example, cells are preferably exposed to nucleic acid and then cultured in the presence of the test agent.

### Neurite Elongation or Degeneration

Methods of the invention may comprise quantifying neuritic growth, neurite elongation or neurite degeneration. Quantifying may comprise determining levels of expression of a neurite-specific protein, wherein a higher level of expression indicates a higher level of neurite growth and/or neurite elongation and/or a lower level of neurite degeneration, and wherein a lower level of expression indicates a lower level of neurite growth and/or neurite elongation and/or a higher level of neurite degeneration. Quantifying may comprise causing or allowing expression of a neuron-specific reporter gene and measuring expression levels of the reporter gene, thereby quantifying neuritic growth, neurite elongation or neurite degeneration. For example, when the reporter gene encodes a fluorescent protein such as GFP, measurement of expression levels comprises measuring fluorescence. Methods of the invention may comprise quantifying neuritic growth, neurite elongation or neurite degeneration by contacting neurons with antibody to a neurite marker (e.g. tubulin, neurofilament, synaptophysin), determining or quantifying antibody binding to the marker, and thereby detecting or quantifying neuritic outgrowth or elongation.

Contacting neurons with antibody may be performed with cell extracts, after lysing cells (e.g. on a Western blot). Alternatively, whole neurons may be contacted with antibody.

Assay methods may comprise culturing neuronal precursor or progenitor or neuronal cells under a first and second condition, respectively, and comparing levels of neurite growth, elongation or degeneration of neuronal precursor or progenitor or neuronal cells cultured under the first condition with neuronal precursor or progenitor or neuronal cells cultured under the second condition, respectively. For example, where levels of neurite growth, elongation or degeneration are higher (e.g. as indicated by increased/decreased level of expression of neurite-specific protein, see above) in the cells cultured under the first condition than in the cells cultured under the second condition, this indicates that the first condition (relative to the second condition) increases neurite growth, elongation or degeneration, respectively.

In a preferred embodiment, culturing under the first condition comprises culturing the cells in the presence of a test compound, wherein the test compound is preferably amyloid β (Aβ) peptide (as derived from amyloid precursor protein, APP).

The invention provides assay methods of identifying an agent that inhibits or reduces an increase in neurite degeneration produced by a condition known to increase neurite degeneration (e.g. wherein the condition is culturing in the presence of amyloid beta peptide), i.e. identifying an agent that reduces or inhibits the effects associated with such a condition. The assay may comprise:
culturing neuronal precursor or progenitor or neuronal cells in the presence of a test agent and under a condition known to increase neurite degeneration;
culturing neuronal precursor or progenitor or neuronal cells in the absence of the test agent and under a condition known to increase neurite degeneration;
quantifying or determining levels of neurite degeneration in the presence and in the absence of the test agent; and
comparing levels of neurite degeneration in the presence of the test agent with levels of neurite degeneration in the absence of the test agent;
wherein a lower level of neurite degeneration in the presence of the test agent compared with the absence of the test agent indicates that the agent inhibits or reduces an increase in neurite degeneration produced by or associated with the condition.

As indicated above, comparing levels of neurite degeneration may comprise comparing levels of expression of neurite specific protein, wherein a higher level of expression (lower level of degradation) in the presence of the test agent compared with the absence of the test agent indicates that the test agent inhibits or reduces an increase in neurite degeneration produced by the condition.

The condition may be the presence of a compound, which may be a compound identified through an assay method of the invention as being able to increase neurite degeneration, Aβ peptide.

### Neuronal Cell Death

A need for neuronal cell death assays exists in the field, and such assays are provided by the present invention.

Neuronal cell death assays may be used to test or determine sensitivity of neurons or a neuronal cell population to a given condition e.g. the presence of one or more compounds, e.g. to identify a condition (e.g. a compound) that increases or reduces neuronal cell death.

For example, an assay according to the present invention may comprise:
culturing neurons under a first condition (“test culture”);
culturing neurons under a second condition (“control culture”);
quantifying or determining neuronal cell death under the first and second conditions; and
comparing levels of neuronal cell death under the first condition with levels of neuronal cell death under the second condition;
wherein a higher level of neuronal cell death under the first condition compared with under the second condition indicates that the first condition increases cell death; and/or
wherein a lower level of neuronal cell death under the first condition compared with under the second condition indicates that the first condition reduces neuronal cell death.

In neuronal cell death assays, especially assays for identifying a condition that reduces neuronal cell death, the neurons are preferably genetically predisposed to apoptosis. For example, the neurons may express p75 neurotrophin receptor, and/or may express an apoptotic protein (e.g. a caspase) operably linked to a neuron-specific promoter (e.g. the Tau locus). Accordingly, ES cells used in the present invention to produce neurons for neuronal cell death assays may express an apoptotic protein (e.g. a caspase) operably linked to a neuron-specific promoter (e.g. the Tau locus).

Neuronal cell death assays may be used to identify an agent that inhibits or reduces an increase in neuronal cell death produced by a condition known to increase neuronal cell death, i.e. an agent that reduces or inhibits the effect of such a condition. The assay may comprise:
culturing neurons in the presence of a test agent and under a condition known to increase neuronal cell death;
culturing neurons in the absence of the test agent and under the condition known to increase neuronal cell death;
quantifying or determining levels neuronal cell death in the presence and in the absence of the test agent; and
comparing levels of neuronal cell death in the presence of the test agent with levels of neuronal cell death in the absence of the test agent;
wherein a lower level of neuronal cell death in the presence of the test agent compared with the absence of the test agent indicates that the agent inhibits or reduces an increase in neuronal cell death produced by the condition.

Cell death may be determined by methods known in the art, for example by determining induction mechanisms of apoptosis in the neurons. Indications of cell death that may be determined include induction of apoptotic proteins (e.g. caspases, especially caspase-3, see ref. 43), staining with propidium iodide and/or DNA fragmentation and/or nucleosome disruption (detectable e.g. by binding of antibody to DNA and/or histone protein, see ref. 44).

### Neurogenesis and Neuronal Differentiation

Methods of the invention may include assays for neurogenesis or neuronal differentiation, wherein production or generation of neurons or differentiation of ES cells and/or neuronal precursor and/or progenitor cells is detected and/or quantified. The method may comprise detecting and/or quantifying one or more neuron-specific markers. Methods of the invention may comprise monitoring levels of neurogenesis for one or more particular neuronal sub-type or lineage, or levels of neurons in general, depending on the markers selected. Generation of neurons of defined lineages may be assayed, by detecting and/or quantifying lineage-specific markers. Methods may comprise contacting the cells with an antibody to a cell marker and determining binding, wherein the presence of the marker (and hence antibody binding) indicates that the cell is of a particular cell type, sub-type, lineage or sub-lineage. Methods may comprise determining or quantifying levels of antibody binding, and thereby determining or quantifying levels of differentiation, the stage of differentiation of the cells, and/or the % cells of a particular type, sub-type, lineage or sub-lineage or at a particular stage of differentiation. More detail on detection of markers and identification of cell types is contained elsewhere herein, and suitable markers are known to the person skilled in the art.

Neuronal differentiation assay methods of the invention are suitable for determining markers that may be used to identify ES cells and/or neural cells at particular stages of differentiation, or to identify the type or sub-type of the cell, and thus indicate the differentiation state of the cell or the cell type or sub-type. For example, assay methods may comprise inducing or allowing differentiation of ES cells to produce neuronal precursor or progenitor cells, and/or culturing neuronal precursor or progenitor cells to produce neurons (preferably using neural differentiation methods as described elsewhere herein); comparing expression levels of proteins in cells at one stage of differentiation with expression levels of proteins in cells at a second stage of differentiation; and identifying proteins whose level of expression differs in cells at the first and second stages of differentiation. A difference in expression levels indicates that the protein may be used as a marker to indicate the differentiation state, type or sub-type of the cell and/or to distinguish cells at the first and second differentiation states. Expression levels may be compared using any appropriate method, which the skilled person can determine. Preferably, expression of proteins expressed at the cell surface is compared, e.g. contacting cells or a cell extract with a surface expression library of antibodies and determining binding. For example, the method may comprise comparing expression of proteins in neuronal precursor/progenitor cells (e.g. radial glial cells) with ES cells.

The difference in expression levels may for example be at least 1.2-fold, at least 1.5-fold, at least 1.6-fold, at least 1.8-fold, at least 2-fold, at least 3-fold, at least 5-fold, at least 10-fold, or more. Expression may be detected in cells at the first stage of differentiation and not detected at all in cells at a second stage of differentiation.

### Electrical Activity

Levels of electrical activity, e.g. electrical activity indicating opening of a specific channel (e.g. ion channel), in the neurons may be observed, detected, determined or quantified.

Assay methods may be used to identify a compound able to modulate electrical activity of neurons. Methods may comprise culturing neurons under a first condition, culturing neurons under a second condition, and comparing electrical activity of neurons cultured under the first condition with electrical activity of neurons cultured under the second condition, respectively. A difference in electrical activity indicates that the condition modulates electrical activity.

### Synaptogenesis

Assay methods may comprise detecting or quantifying synaptogenesis in neuronal cells. Detecting or quantifying may comprise measuring electrophysiological activity of the cells and/or detecting or measuring expression of one or more markers indicative of synaptogenesis e.g. synaptophysin.

### Comparison of Genetically Distinct Neurons

The present invention provides methods of comparing a reference (normally wild-type) neuronal precursor or progenitor cell or neuron with a mutant neuronal precursor or progenitor cell or neuron, the neurons having different genotypes. The method may include a method described above for producing neural cells.

Accordingly, the present invention provides a method comprising:
providing a first and a second culture of neuronal cells or neuronal precursor or progenitor cells, wherein cells in the first culture have a different genotype to cells in the second culture; and
comparing neuronal precursor or progenitor cells or neurons in the first culture with neuronal precursor or progenitor cells or neurons in the second culture.

Neuronal precursor or progenitor or neuronal cells with and without a mutation in a gene of interest may be compared. The mutation may for example be deletion of all or part of the gene, deletion of all or part of the gene promoter and/or enhancer, or substitution of one or more nucleotides in the coding region, promoter or enhancer. Normally, the mutation results in an altered (reduced or increased) level of expression of the gene, or in expression of a mutated protein (e.g. truncated or containing one or more deletions or substitutions in its amino acid sequence). Alternatively, neuronal precursor or progenitor or neuronal cells in the first culture may contain an introduced gene (e.g. an inserted gene or inserted cDNA) or overexpress an endogenous gene, whereas neuronal precursor or progenitor or neuronal cells in the second culture do not.

ES cells may be genetically manipulated and mutations may be induced in ES cells, or ES cells may be isolated from an animal carrying a mutation, e.g. a mouse ES cell having a mutation of interest. Methods of the present invention may use ES cells with and without the mutation of interest, to generate neural cells e.g. neurons or neuronal progenitor/precursor cells with and without the mutation of interest, respectively. Thus, the present invention may produce mutant and wild-type neural cells, e.g. neurons or neuronal progenitor/precursor cells. Comparison between neural cells produced from different ES cell types (one having a mutation of interest, the other not) may for example be performed to identify a mechanism responsible for or contributing to loss of a neural cell type in a neurodegenerative disease, and to identify relevant targets in disease phenotypes.

In some embodiments, the method may comprise producing neuronal precursor/progenitor cells or neurons from a first and a second culture of ES cells, respectively, wherein ES cells in the first and second cultures have different genotypes. Optionally, the neuronal precursor/progenitor cells or neurons may be produced from ES cells by methods of the invention as described elsewhere herein. ES cells in the first culture may contain a mutation in a gene of interest, while ES cells in the second culture do not contain the mutation (e.g. wild-type cells). Alternatively, ES cells in the first culture may contain an introduced gene or overexpress an endogenous gene, whereas ES cells in the second culture do not.

As an alternative to using genetically distinct ES cells, dissociated EB cells may be genetically manipulated. Methods may comprise transfecting a first culture of dissociated EB cells, or neuronal precursor or progenitor cells, with a nucleic acid construct, thereby changing the genotype of cells in the first culture compared with cells in the second culture. For example, the nucleic acid construct may encode an endogenous gene, or encode a gene of interest containing a mutation. Such methods of the invention normally comprise allowing expression (normally transient expression, lasting about 2, 3 or 4 days) from the nucleic acid construct. The method may comprise culturing the cells to produce neuronal cells. Cells in the first culture would be compared with neuronal precursor, progenitor or neuronal cells in a second culture, wherein the cells in the second culture do not contain the nucleic acid construct, introduced gene and/or mutation.

Comparing neuronal precursor or progenitor cells or neurons may comprise comparing (and normally determining or quantifying) one or more characteristics such as neuritic growth or neurite elongation, neuronal shape, neuronal cell death, neurogenesis, neuronal differentiation, electrical activity, synaptogenesis and/or neuronal cell markers. In other embodiments, comparing may comprise comparing readout of the gene of interest e.g. the introduced or mutated or overexpressed gene, or the effects of that gene. The nature of the readout depends on the gene, but can be determined by the skilled person for a given gene. Thus, neuronal signalling mechanisms may be clarified, blocked and/or manipulated.

Comparing neuronal precursor or progenitor or neuronal cells may comprise comparing one or more characteristics of the cells under a test condition, and methods of comparing genetically distinct neuronal precursor or progenitor cells or neuronal cells may be used in the context of assay methods described elsewhere herein. Thus, in preferred embodiments, the first and second cultures of cells are each cultured under a test condition, and neuronal characteristics of the cells are compared. Further methods and variations are as described above for cellular assays. For example, culturing under the test condition may comprise culturing in the presence of Aβ peptide.

### Antibodies

As used herein, “antibody” or “antibodies” covers any specific binding substance or substances having a binding domain with the required specificity. Thus, this term covers antibody fragments, derivatives, functional equivalents and homologues of antibodies, including any polypeptide comprising an immunoglobulin binding domain, whether natural or synthetic. Chimaeric molecules comprising an immunoglobulin binding domain, or equivalent, fused to another polypeptide are therefore included. Cloning and expression of chimaeric antibodies are described in EP-A-0120694 and EP-A-0125023.

It has been shown that fragments of a whole antibody can perform the function of binding antigens. Examples of binding fragments are (i) the Fab fragment consisting of VL, VH, CL and CH1 domains; (ii) the Fd fragment consisting of the VH and CH1 domains; (iii) the Fv fragment consisting of the Vl and VH domains of a single antibody; (iv) the dAb fragment (Ward, E. S. et al., Nature 341, 544-546 (1989) which consists of a VH domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al, Science, 242, 423-426, 1988; Huston et al, PNAS USA, 85, 5879-5883, 1988); (viii) bispecific single chain Fv dimers (PCT/US92/09965) and (ix) “diabodies”, multivalent or multispecific fragments constructed by gene fusion (WO94/13804; P Holliger et al Proc. Natl. Acad. Sci. USA 90 6444-6448, 1993).

Diabodies are multimers of polypeptides, each polypeptide comprising a first domain comprising a binding region of an immunoglobulin light chain and a second domain comprising a binding region of an immunoglobulin heavy chain, the two domains being linked (e.g. by a peptide linker) but unable to associate with each other to form an antigen binding site: antigen binding sites are formed by the association of the first domain of one polypeptide within the multimer with the second domain of another polypeptide within the multimer (WO94/13804).

Antibodies may be modified in a number of ways, e.g. they may be labelled, for example with a fluorescent label allowing antibody binding to be quantified by measuring levels of fluorescence.

Various further aspects and embodiments of the present invention will be apparent to those skilled in the art in view of the present disclosure.

All documents mentioned in this specification are incorporated herein by reference in their entirety.

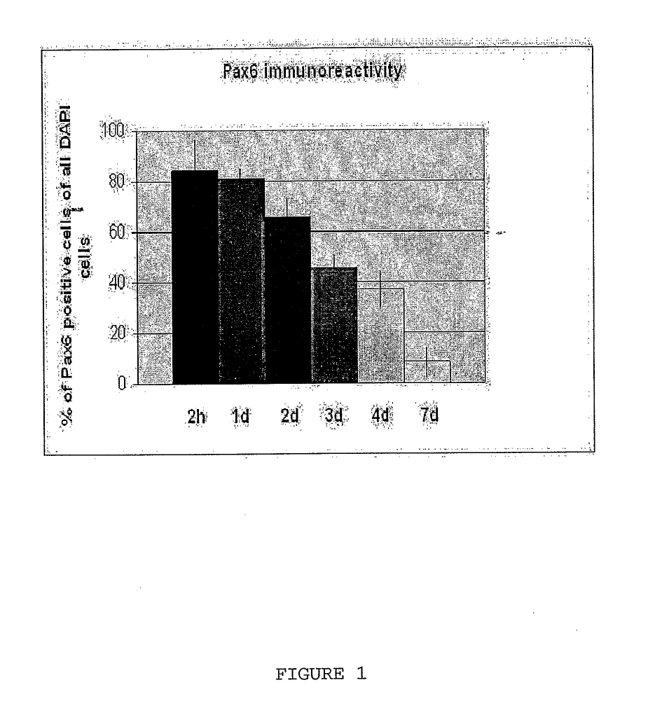

Certain aspects and embodiments of the invention will now be illustrated by way of example only and with reference to FIG. 1, which shows % Pax6 positive cells at selected time points after plating dissociated EB cells. Pax-6 is initially expressed by most cells but rapidly disappears. The results represent the mean of 4 independent experiments performed with 2 different ES cell lines. They are expressed as % ±SD with 100% as the number of DAPI-positive nuclei.

### EXAMPLES

### Culture of ES Cells

The procedure leading to the generation of neurons from ES cells involved the following steps, summarised as follows:

1. Cells cultured on a feeder layer grow as colonies while after feeder-deprivation they grow as a flat monolayer.
2. ES cells on non-adherent bacterial dishes form cellular aggregates (EBs) that grow in suspension.
3. After 4 days of EB formation, RA was added for another 4 days.
5. EBs were dissociated after a total of 8 days and plated onto PDL/laminin-coated dishes in N2 medium.
6. N2 medium was changed after 2 h and again after 12-24 h. At this stage most precursor cells have a spindle-shape morphology. The neuronal differentiation medium is added after 30-48 h.

This procedure was developed using ES cells expressing GFP from the tau locus (ref. 13). Expression of GFP from an endogenous promoter allowed visualisation of neurons and of their processes under UV light, and we used it to maximise the generation of fluorescent cells.

After thawing, ES cells were first cultured on feeder cells for 2 to 3 passages and then progressively deprived of feeder cells. Defined numbers (3×10⁶) of cells were then used to form aggregates (embryoid bodies, EBs) that were incubated in non-adhesive bacterial dishes (10 cm dish, 15 ml medium) for 8 days. Retinoic acid (RA, 5 μM) was added after 4 days and left for the last 4 days. An important step was the selection of feeder-deprived ES cells with a homogenous, flat morphology and a high proliferation rate (see Materials and Methods).

After 8 days, the EBs were dissociated with a freshly prepared suspension of trypsin and plated on a substrate consisting of poly-D-lysine (PDL) and laminin. The plating density (1.5×10⁵ cells/cm²) was found to be crucial as at lower densities cells tended to die rapidly. The dissociated cells were plated in serum-free medium that was changed 2 h after plating to remove debris and dead cells. Medium was changed again after one day (roughly 24 hours). After 48 h the medium was replaced by a serum-free medium enriched with supplements (ref. 12). In addition to ES cells expressing GFP from both tau alleles, we also used over 7 other ES cell lines with results that were indistinguishable from those reported in this study. These include wild-type J1 and E14 ES cells, as well as J1 with GFP in one or both of the tau alleles. We also isolated 4 different ES cell lines from blastocysts of mixed BL6/SV129 backgrounds and submitted them to our differentiation protocol with similar results.

### Neuronal Precursors

The ES cells differentiated into a homogeneous population of radial glial cells.

Cells dissociated from the EBs adopt a distinct elongated, spindle-shaped morphology reminiscent of the shape of radial glial cells (see ref. 16). Phase-contrast image illustrated bipolar spindle-morphology at 2 hours of differentiation.

These cells were identified as neural precursor cells by staining with an antibody to the intermediate filament protein nestin (ref. 9). Two hours after plating, the vast majority of the cells were found to be positive for nestin when compared with the total number of plated cells quantified by nuclear staining (Table I).

We then used RC2, a marker expressed by all radial glial cells, and nearly all cells were found to be positive (Table I). Staining with antibodies to brain lipid binding protein (BLBP), an antigen that is also expressed by radial glial cells in the developing CNS (ref. 18), further confirmed the identity of the cells freshly dissociated from EBs (Table I). The homeodomain transcription factor Pax-6 is expressed by all cortical radial cells (ref. 19) and essentially all cells in EBs were found to express it before their dissociation, which means that at this stage they are already precursors.

Quantification 2 h after plating revealed that the vast majority of the cells were still positive for Pax-6 (FIG. 1), and that its expression rapidly decreased over the following days to be essentially absent after 7 days (FIG. 1).

TABLE 1

| Nestin | RC2 | BLBP | Pax6 |
|---|---|---|---|
| 88 ± 2.5 | 99 ± 2.5 | 97 ± 2.6 | 84 ± 12.2 |

% neuronal precursors 2 hours after plating. Nestin, RC2, BLBP, Pax-6 were analyzed by immunocytochemistry 2 hours after plating the dissociated EBs. The percentage (±SD) of positive cells was determined in relation to the total number of cells stained by the nuclear marker DAPI.

### Neuronal Differentiation

Cells with neuronal morphology begun to appear within less than 2 days following EB dissociation. All differentiating cells expressed GFP indicating that they were neurons, a conclusion supported by staining experiments in which an antibody was used that recognizes a neuron-specific form of tubulin.

After 4 days, about 85% of the cells were GFP- and tubulin-positive. Both by phase contrast and fluorescence we were struck by the remarkably homogeneous appearance of the neuronal cell bodies. With time in culture they increasingly adopted the pyramidal shape observed with cells isolated from the rodent hippocampus (ref. 20). When stained with antibodies to synaptophysin, numerous clusters were seen lining GFP-positive processes, indicating that synaptic contact may develop in our cultures.

To test if these neurons use glutamate as neurotransmitter, we stained the cells with an antibody to the vesicular glutamate transporter, vGlut1, a membrane protein expressed by most pyramidal neurons in the cerebral cortex and in the hippocampus (ref. 21). After 7 days in culture, 93±4.7% of the cells were stained with vGlut1 antibodies. The findings with vGlut1 antibodies are consistent with the identification of the neurons as pyramidal cells. At the end of the first week following EB dissociation, fewer than 0.1% of the cells stained positive for Isl-1, tyrosine hydroxylase and choline acetyltransferase. Less than 5% were GABA positive after 3 weeks.

To identify proteins expressed during the transition from radial glial cells to neurons, we performed Western blot analyses using in vitro differentiated neurons prepared at different time intervals.

While undetectable in lysates of radial glial cells, the AMPA receptor subunit GluR1, like synaptophysin, was clearly detectable after a few days of culture. GluR1 and synaptophysin protein levels increased as neurons began to differentiate.

As pyramidal neurons express high levels of Trk receptors, both in the cerebral cortex and in the hippocampus (ref. 22), we also analyzed their expression using an antiserum directed against the intracellular domain of these neurotrophin receptors. While Trk receptors were hardly detectable at day 5, their levels increased dramatically over the following days Substantial expression of Trk receptors was observed after about 7 days in vitro, after which time it increased dramatically. Conversely, the levels of the neurotrophin receptor p75 were found to decline during the course of neuronal maturation, much like they do in vivo (ref. 23).

Finally, we tested the expression of the amyloid precursor protein (APP). This membrane protein has been shown to be expressed by radial glial cells (ref. 24), as well as by a number of cells including neurons later in development. Our results demonstrate that unlike other membrane proteins tested, APP is clearly detectable in lysates of radial glial cells. The levels of expression subsequently increase, presumably as a consequence of neuronal maturation that includes a marked growth of neuronal processes.

### In Vivo Differentiation of Implanted Precursors

The developmental potential of the neuronal precursor cells of the invention was tested by implanting them in chick embryos where they could differentiate into different specific neuronal lineages, including motoneurons.

### Electrophysiology

Electrophysiological experiments showed that the neurons formed synapses, showed APs and were very homogeneous in electrophysiological characteristics. The neurons were mainly glutamatergic (shown by blocking of synaptic currents with NBQX, vGAT staining) with some gabaergic input (blocking with bicuculline, otherwise the culture would not survive). Electrophysiology clearly showed that no other neuronal cell types were present under the conditions used.

To characterise the electrophysiological properties of our ES cells-derived neurons, whole-cell patch-clamp recordings were performed on cells that had been in culture for 10 and up to 22 days. All cells investigated (n=22) showed spontaneous or depolarization-induced action potentials and in all cases these could be blocked by the application of tetrodotoxin. Also, the electrophysiological characteristics of the cells investigated indicated that they were fairly homogeneous with regard to their functional properties which were similar to those previously described for pryamdial neurons. Spontaneous synaptic currents (SSCs) could be observed which could be completely blocked by the addition of NBQX/AP-5 and bicuculline, or of NBQX/AP-5 alone. These results indicate that the ES cells-derived neurons form functional synapses that utilise glutamate as neurotransmitter. As these experiments also revealed the presence of functional GABA-synapses in long-term cultures, we quantified the number of GABA-neurons after 3 weeks. With antibodies to the vesicular transporter vGAT we found that about 5% of the cells were positive for this marker after 3 weeks. Consistent with the lack of staining for neurotransmitters unrelated to the glutamate and GABA system, we did not detect any synaptic activity that could not be attributed to glutamate or GABA.

### Discussion

Using mouse ES cells, we found conditions leading to the generation of a virtually pure population of neuronal precursors defined as radial glial cells. These cells then go on to generate a homogeneous population of neurons with the characteristics of pyramidal cells.

When highly proliferative, uncommitted stem cells are selected for the formation of EBs, we found that treatment with RA converts the entire cell population into a defined type of neuronal precursor. The selection of uncommitted ES cells is important as it has been observed that even in the presence of LIF, some ES cells have a tendency to differentiate and that during EB formation, cells of different lineages can often be observed (for reviews, see ref. 3, 34).

To select for highly proliferative ES cells following the progressive removal of feeder cells, we monitored the rate of division by cell counting, the phase contrast appearance of the cells as well as the degree of confluency they reach before starting EB formation with a defined number of cells.

The presence of radial glial cells in non-dissociated EBs has already been reported using either ES cells or P19 embryonic carcinoma cells (ref. 35). When EBs were plated on a polylysine substrate, elongated cells could be observed migrating radially, away from the EBs and to progressively transform into astrocytes (ref. 35). The identification of the cells we obtained by dissociating EBs is based on their morphology and their quantification on staining with RC2-, BLBP- and Pax-6-antibodies. This set of markers has previously been shown to be expressed by radial glial cells in the cortex (ref. 11,16). Of note is the fact that not all radial glial cells express Pax-6. In particular, those located in the ganglionic eminence do not express Pax-6 and are not neurogenic (ref. 11). Interestingly, it has recently been demonstrated that the addition of RA to EBs leads to the induction of the Wnt signaling antagonist sFRP2 (ref. 36). It is then conceivable that inhibition of Wnt signalling by molecules present in the developing forebrain causes cells to adopt a radial glial cell phenotype. The spatial and temporal expression pattern of sFRP1 is compatible with this view (ref. 37).

Under our in vitro conditions, the addition of RA is crucial (ref. 38). While after 4 days of RA treatment virtually all cells express Pax-6 in EBs, no Pax-6-positive cells could be observed in the absence of RA and no neurons were obtained following the dissociation of untreated EBs. While it appears unlikely that RA plays a physiological role on the induction of Pax-6 in the developing cortex, this may well be the case in other parts of the developing CNS. Indeed, while Pax-6 has a restricted pattern of expression in the CNS that includes the cerebral cortex, it is also expressed in much of the ventral neural tube during development and recent results suggest that somite-derived RA plays a physiological role in the ventral patterning of the neural tube (ref. 39, 40). With regard to RA-treated EBs, Renoncourt et al. (ref. 28) and Wichterle et al. (ref. 7) also observed that some cells in the EBs were Pax-6-positive following RA treatment. However, Pax-7-positive cells were also observed with similar abundance (ref. 7), suggesting heterogeneity in the cellular composition of the RA-treated EBs.

On a polycationic substrate coated with laminin, the radial glial cells rapidly lose their typical spindle shape morphology. When using our EGP-ES lines, we noted that the number of fluorescent cells rapidly increased and that neurons all looked remarkably similar with regard to their shapes and the size of their cell bodies. By the 4th day following dissociation, virtually all cells already had neuronal characteristics. With increasing time, essentially all neurons adopted a pyramidal shape and were also found positive for a glutamate vesicular transporter. All cells were stained at all times irrespective of their identity. By contrast less than 0.1% of the cells were clearly positive when stained after 1 week in culture with antibodies to Isl-1, tyrosine hydroxylase or choline acetyl transferase. Less than 5% were positive for vGAT after three weeks. The absence of GABA and of Isl-1 staining rules out a number of interneurons and long projection neurons, including in particular motoneurons, many of which also derive from Pax-6 positive cells in vivo.

Presumably, inductive signals such as sonic hedgehog need to be present to drive the progeny of Pax-6-positive radial glial cells along this particular differentiation pathway (ref. 7). The glutamaergic phenotype of our neurons is consistent with their identity as cortical pyramidal neurons, as their shape indicates. Most importantly, this indication is in line with the observation that these neurons all derive from radial glial cells. Indeed, Malatesta et al. (ref. 11) recently demonstrated that the progeny of cortical radial glial cells are pyramidal neurons populating all cortical layers, as well as the hippocampus. Our culture conditions could thus be described as being “permissive”, allowing a differentiation program intrinsic to radial glial cells to unfold in vitro. In line with this, the medium we used was initially-developed to support the survival and differentiation of pyramidal neurons isolated from the embryonic rodent hippocampus (ref. 12). A property of this medium is also to prevent or repress the multiplication of cells such as astrocytes. These cells would be expected to be present in our cultures since they also belong to the progeny of radial glial cells.

Using GFAP antibodies, we did observe the development of a few ramified astrocytes in our culture. However, their numbers were very small (in the range of 1-2% of the total number of cells after 3 weeks).

The relative uniformity of our neuronal cultures prompted us to examine the expression of membrane proteins known to be expressed at specific developmental time points. In line with in vivo results (ref. 23), we found that p75 expression is tightly correlated with the appearance of neurons in our cultures and is subsequently down-regulated. By contrast while Trk receptor expression is undetectable at early time points, it increases dramatically after a few days, suggesting that the neurons develop in synchrony. High levels of Trk receptor expression is a characteristic of pyramidal neurons in vivo (ref. 22). RT PCR experiments suggest that both TrkB and TrkC contribute to the signal obtained using pan-Trk antibodies, while TrkA expression is barely detectable after the first few days in vitro. By contrast with p75 and Trk receptors, APP is clearly detectable already 2 h after EB dissociation and its levels increase during the course of neuronal differentiation. This is in line with the results of immunohistochemistry experiments indicating that APP specifically labels radial glial cells in the developing rodent cortex (ref. 24).

### Materials and Methods

### Material

ES cell culture medium ingredients were obtained from Gibco, LIF was from Chemicon, PDL and stocks for N2 and complete medium from Sigma. BSA powder fraction V was from Gibco. Laminin was isolated from Engelbreth-Holm-Swarm sarcoma (Roche). RA was obtained from Sigma and no differences in the results were observed when using different batches.

### Antibodies

Primary antibodies for immunocytochemistry were mouse monoclonal antibody antinestin (rat401, IgG1; 1:10; Developmental Studies Hybridoma Bank, DSHB), mouse monoclonal antibody RC2 (IgM; 1:4; DSHB), rabbit polyclonal antibody anti-BLBP (1:2000; kindly provided to M. Goetz by N. Heintz, Rockefeller University, New York), mouse monoclonal antibody anti-Pax6 (IgG1; 1:100; DSHB), mouse monoclonal antibody anti-βtubulinIII (IgG2b; 1:100; Sigma) and rabbit polyclonal antibody anti-vGlut1 (1:5000; SYSY). Subclass-specific Cy2- or Cy-3-coupled antisera were used as secondary antibodies. For Western Blotting we used mouse monoclonal antibody anti-synaptophysin (IgG1; 1:1000; Sigma), rabbit polyclonal antibody anti-GluR1 (1:1000; Upstate), rabbit polyclonal antibody anti-Trk (C-14, sc-11; 1:1000; Santa Cruz), rabbit polyclonal antibody anti-APP (1:3000; kindly provided by P. Paganetti, Novartis, Basel) and rabbit polyclonal antibody anti-p75 (1:2000; Promega).

Media
ES Medium (500 ml):

| | | |
|---|---|---|
| DMEM | 410 | ml |
| FCS | 75 | ml (heat inactivated |
| | | 55° C. 30 min) |
| LIF | 5 | ml |
| Glutamine | 5 | ml |
| Non-essential | 5 | ml |
| amino acids | | |
| β-MeOH | 5 | μl |

EB Medium (500 ml):

| | | |
|---|---|---|
| DMEM | 440 | ml |
| FCS | 50 | ml |
| Glutamine | 5 | ml |
| Non-essential | 5 | ml |
| amino acids | | |
| β-MeOH | 5 | μl |

N2 medium:

| | | | | |
|---|---|---|---|---|
| DMEM | 125 | ml | | |
| Glutamine | 1.25 | ml | | |
| F-12 (Gibco #21765029) | 125 | ml | | |
| Insulin | 1.25 | ml | 25 | μg/ml |
| Transferrin | 6.25 | ml | 50 | μg/ml |
| Progesterone | 0.25 | ml | 6 | ng/ml |
| Putrescine | 0.25 | ml | 16 | μg/ml |
| Sodium selenite | 25 | μl | 30 | nM |
| BSA | 1.25 | ml | 50 | μg/ml |

| | | | |
|---|---|---|---|
| P/S | 2.5 | ml | 1% |

P/S represents antibiotic e.g. penicillin/streptomycin. It may optionally be excluded from media herein and replaced by equivalent volume of DMEM.

### Stock Solutions for N2 Medium:

### BSA Gibco #A-9418 Powder Fraction V 100 g

Aliquots 10 mg/ml stored at −20° C.
Final conc. 50 μg/ml

### Insulin Sigma I-6634 100 mg

Stock solution 5 mg/ml in H₂O (acidified with a drop concentrated HCl to pH 2 to dissolve the insulin)

### Stored at −80° C.

Transferrin Sigma #T-1147 apo-transferrin human 100 mg
Stock solution 2 mg/ml in H₂O

### Stored at −80° C.

### Progesterone Sigma #P-8783 5 g

Stock solution 2 mM in EtOH stored at −80° C.
Working solution 20 μM dilution of stock solution in H₂O stored at −80° C.

### Putrescine Sigma #P-5780

Stock solution 100 μM in H₂O stored at −80° C.
Sodium selenite Sigma #S-5261 25 g
Stock solution 300 μM in H₂O stored at 4° C.

### Complete Medium:

Aqueous solutions:

| | | |
|---|---|---|
| L-Alanin (Sigma #A-7627) [Stock solution 2 mg/ml] | 2 | μg/ml |
| Biotin (Sigma #B-4501) [Stock solution 0.1 mg/ml] | 0.1 | μg/ml |
| L-Carnitine (Sigma #C-0283) [2 mg/ml] | 2 | μg/ml |
| Ethanolamine (Sigma #E-9508) [1 mg/ml) | 1 | μg/ml |
| D+-Galactose (Sigma #G-0625) [15 mg/ml] | 15 | μg/ml |
| L-Proline (Sigma #P-0380) [7.76 mg/ml] | 7.76 | μg/ml |
| Putrescine (Sigma P-7505) [16.1 mg/ml] | 16.1 | μg/ml |
| Na-Pyruvate (Sigma #P-5280) [25 mg/ml] | 25 | μg/ml |
| Na-Selenite (Sigma #S-1382) [0.016 mg/ml] | 0.016 | μg/ml |
| Vitamine B12 (Sigma #V-2876) [0.34 mg/ml] | 0.34 | μg/ml |
| Zinc sulfate (Sigma #Z-4750) [0.194 mg/ml] | 0.194 | μg/ml |
| Catalase (Sigma #C-40) [16 mg/ml] | 16 | μg/ml |
| Glutathione (Sigma #G-6013) [1 mg/ml] | 1 | μg/ml |
| SOD (Sigma #S-2515) [2.5 mg/ml] | 2.5 | μg/ml |

Ethanolic solutions:

| | | |
|---|---|---|
| Linoleic acid (Sigma #L-1376) [100 mg/ml] | 1 | μg/ml |
| Linolenic acid (Sigma #L-2376) [100 mg/ml] | 1 | μg/ml |
| Progesterone (Sigma #P-8783) [0.63 mg/ml] | 6.3 | ng/ml |
| all trans Retinol (Sigma #R-7632) [10 mg/ml] | 100 | ng/ml |
| Retinylacetate (Sigma #R-7882) [10 mg/ml] | 100 | ng/ml |
| Tocopherol (Sigma #T-3251) [100 mg/ml] | 1 | μg/ml |
| Tocopherolacetate (Sigma #T-3001) [100 mg/ml] | 1 | μg/ml |

### Dissolve

| | | |
|---|---|---|
| BSA | 1 | g |
| Transferrin | 2 | mg |
| Insulin | 1.6 | mg |
| Glutamine | 2 | mM |

| | |
|---|---|
| P/S (optional) | 1% |

in 400 ml DMEM and add the above solutions.

### ES Cell Culture

Initially ES cells were cultured on feeder cells consisting of mitomycin-inactivated mouse embryo fibroblasts for at least two passages after thawing. For the following passages ES cells were cultured without feeder cells and differentiation could either be started immediately after at least two passages without feeder cells or from frozen stocks of feeder-free ES cells. Stocks used for differentiation were passaged at least twice before starting the procedure. After culture of ES cells on feeder cells the first passage without feeders was important. Successful differentiation depended on the density of the ES cells used for this first passage. ES cells should occupy at least one third of the plate 1 day after splitting. ES medium was based on DMEM containing 15% FCS (specifically tested for ES cell culture followed by neuronal differentiation), LIF (1000 U/ml), non-essential amino acids and β-mercaptoethanol. Cell culture plates were always coated with a 0.2% gelatin solution for at least 10 min. The temperature of incubation was found to be an important factor as neuronal differentiation was not successful above 37° C. ES cells were maintained at a maximal temperature of 37° C. in 7% CO₂/air atmosphere. All media were prewarmed at 37° C.

ES cells were split every 2 days with plating densities between 1.5×10⁶ and 4×10⁶ cells on 10 cm cell culture plates (Corning). After 2 days between 10-25×10⁶ cells can be recovered and a high proliferation rate is a necessary condition for the success of the experiment. The cells have to be in a phase of rapid growth and form a flat monolayer.

Splitting of cells is done by 2×PBS wash and incubation of the cells with a thin film of trypsin solution (1× solution trypsin Gibco—0.05% in 0.02% EDTA) at 37° C. 7% CO₂ for 3 min, plates can be shaken by hand and cells will come off and be resuspended in fresh ES medium by pipetting up and down (inactivation of trypsin). Centrifugation follows for 5 min at 1000 rpm room temperature. The pellet is resuspended again in fresh ES medium by pipetting up and down several times. The cells should be dissociated to a single cell culture, although aggregates of 2-3 cells may be present; larger clumps should not occur. The desired amount of cells is re-plated on gelatine-coated plates.

To deprive ES cells of feeders they can be cultured after thawing approximately twice on feeders and then at least 2 passages without feeder cells will be performed so that fibroblasts become diluted out. ES cell thereby change from a colony-like shape to a flat morphology.

Thawing ES cells involves thawing a stock vial of about 3×10⁶ ES cells quickly, resuspending the cells in 10 ml ES medium and centrifuging for 5 min at 1000 rpm room temperature. The cell pellet is resuspended in ES medium again and the amount of cells is plated to a 6 cm cell culture dish. Freezing ES cells is done by resuspending the cells when splitting after trypsination and centrifugation in ES medium+10% DMSO.

### Neuronal Differentiation Protocol.

For EB formation, 3×10⁶ ES cells were plated onto non-adherent bacterial dishes (Greiner) in 15 ml EB medium (ES medium without LIF and only 10% FCS) and incubated for 8 days.

Medium was changed every 2 days by removing the total cell culture from the bacterial dish (in a 50 ml Falcon tube) and letting the EBs settle down (about 3-5 min). The supernatant is then carefully sucked off, and EBs are recovered in 15 ml EB medium again. EBs should be carefully resuspended in medium by pipetting, using a pipette with a sufficiently wide opening to avoid damaging or dissociating the EBs (e.g. 10 ml plastic pipette).

RA (Sigma), 5 μM, was added after 4 days directly to the dish and dispersed by shaking the plate softly. RA should not be left too long under light as it is light-sensitive. EBs were then dissociated and the cells plated on PDL/Laminin coated plates, as follows.

Cell culture dishes were coated with a solution of 10 μg/ml PDL solution in borate buffer (150 mM pH 8.4) and placed overnight (37° C., 7% CO₂) in the incubator. Polyornithine was also used at 100 μg/ml with similar results. After washing the plates three times with PBS (H₂0 in the case of polyornithine), laminin (approx 0.5 μg/cm²) was added directly to the PBS solution and the plates returned to the incubators for at least 2 h.

After 8 days of EB formation, EBs were washed 2× with PBS and trypsinized by incubating them 3 min in a water bath at 37° C. in a 0.05% trypsin solution in 0.04% EDTA/PBS (freshly prepared with trypsin powder, TPCK-treated, Sigma). During the incubation time the Falcon tube should be shaken carefully by hand a couple of times and disintegration of the EBs can be seen easily. Dissociated EBs were then gently, but thoroughly resuspended in 10 ml EB medium containing serum for trypsin inactivation. Dissociation can be done by approx. 5× pipetting up and down. The best trituration was by a smooth edged/in flame Pasteur pipette with a small volume (about 1.5 ml) 2× and then with a 5 ml plastic pipette. Trituration was followed by 5 min centrifugation with 1000 rpm at room temperature. Supernatant was then removed entirely, the pellet was resuspended in N2 medium, and the cell suspension was filtered through a 40 μm Nylon cell strainer (Falcon).

Laminin was removed from the coated plates, and the cell suspension added immediately, without allowing the plates to dry. Dissociated cells were plated at a density of 1.5×10⁵ cells/cm². The N2 medium was changed after 2 h and again after 1 d. After 2 d the medium was replaced by the enriched, serum-free medium described by Brewer and Cotman (ref. 12) with the modification that glutamate, HEPES, corticosterone, lipoic acid and T3 were omitted.

Neuronal differentiation continues and neuronal cultures can be maintained for several weeks.

### Immunocytochemistry

Glass coverslips were prepared by washing them in water and incubating in 65% nitric acid for 1 to 2 days. Subsequently, they were floated in H₂0 for several hours, rinsed in ethanol, air-dried and sterilized under UV light. Cells were fixed with 4% paraformaldehyde (PFA) for 10 minutes, washed in PBS and blocked for 1 h in blocking buffer (0.03% carrageenan, 10% NGS, 0.3% Triton X-100). Mounting was in AquaPoly/Mount (Polysciences).

### Western Blotting

Dissociated EBs were plated as indicated above and samples for Western Blots were collected at the indicated time points. Plates were washed twice with ice-cold PBS before harvesting. Whole cell extracts were prepared in 750 μl lysis buffer for a 6 cm plate (50 mM Tris pH 7.4, 150 mM NaCl, 10% glycerol, 1% Triton X-100) supplemented with protease inhibitor cocktail (Roche). After centrifugation for 30 min at 4200 rpm in an Eppendorf centrifuge, the supernatant was removed and the protein content determined by DC Protein Assay (BioRad). Samples were boiled in Laemmli buffer and 5 μg were loaded onto polyacrylamide gels. Blots were blocked with a 5% milk solution, incubation was overnight with the primary antibody and 2 h with the secondary antibody. Detection was performed with ECL Plus (Amersham).

### REFERENCES

1. Evans, M. J. & Kaufman, M. H. Nature 292, 154-156 (1981).
2. Martin, G. R. Proc. Natl. Acad. Sci. USA 78, 7634-7638 (1981).
3. Stavridis, M. P. & Smith, A. G. Biochem. Soc. Trans. 31, 45-49 (2003).
4. Rathjen, J. & Rathjen, P. D. Curr. Opin. Genet. Dev. 11, 587-594 (2001).
5. Chung, S. et al. Eur. J. Neurosci. 16, 1829-1838 (2002).
6. Kawasaki, H. et al. Neuron. 28, 31-40 (2000).
7. Wichterle, H. et al. Cell 110, 385-397 (2002).
8. Kondo, M. et al. Annu. Rev. Immunol. 21, 759-806 (2003).
9. Lendahl, U., Zimmerman, L. B. & McKay, R. D. Cell 60, 585-595 (1990).
10. Li, M., et al. Curr. Biol. 8, 971-974 (1998).
11. Malatesta, P. et al. Neuron 37, 751-764 (2003).
12. Brewer, G. J. & Cotman, C. W. Brain Res. 494, 65-74. (1989)
13. Tucker, K. L., Meyer, M. & Barde, Y. A. Nat. Neurosci. 4, 29-37 (2001).
14. Bain, G., Kitchens, D., Yao, M., Huettner, J. E. & Gottlieb, D. I. Dev Biol. 168, 342-357 (1995).
15. Li, M. in Methods in Molecular Biology: Lineage selection for generation and amplification of neural precursor cells. Embryonic Stem Cells: Methods and Protocols 185, 205-215 (Humana Press Inc., Totowa, N.J., 2002)
16. Hartfuss, E., Galli, R., Heins, N. & Götz, M. Dev. Biol. 229, 15-30 (2001).
17. Misson, J. et al. Brain Res. Dev. Brain Res. 44, 95-108 (1988).
18. Feng, L., Hatten, M. E. & Heintz, N. Neuron 12, 895-908 (1994).
19. Götz, M., Stoykova, A. & Gruss, P. Neuron 21, 1031-1044 (1998).
20. Banker, G. A. & Cowan, W. M. Brain Res. 126, 397-442 (1977).
21. Fremeau, R. T. Jr et al. Neuron 31, 247-260 (2001).
22. Klein, R. et al. Development 109, 845-850 (1990).
23. Bothwell, M. Annu. Rev. Neurosci. 18, 223-53 (1995).
24. Trapp, B. D. & Hauer, P. E. J. Neurosci. Res. 37, 538-550 (1994).
25. Fraichard, A. et al. J. Cell Sci. 108, 3181-3188 (1995).
26. Strübing, C. et al Mech. Dev. 53, 275-287 (1995).
27. Okabe, S., Forsberg-Nilsson, K., Spiro, A. C., Segal, M. & McKay, R. D. Mech. Dev. 59, 89-102 (1996).
28. Renoncourt, Y. et al. Mech. Dev. 79, 185-197 (1998).
29. Rathjen, J. et al. Development 129, 2649-2661 (2002).
30. Abe, Y. et al. J. Neurosci. 23, 8513-8525 (2003).
31. Barberi, T. et al. Nat. Biotechnol. 21, 1200-1207 (2003).
32. Jones-Villeneuve, E. et al. J. Cell Biol. 94, 253-262 (1982).
33. Bain, G., et al. Biochem. Biophys. Res. Commun. 223, 691-694 (1996).
34. Murray, P. & Edgar, D. Proc. Roy. Soc. Meeting Review (in press).
35. Liour, S. S. & Yu, R. K. Glia 42, 109-117 (2003).
36. Aubert, J., et al. Nat Biotechnol. 20, 1240-1245 (2002).
37. Kim, A. et al. Mech. Dev. 103, 167-172 (2001).
38. Gajovic, S., et al. Differentiation 62, 187-192 (1997).
39. Diez del Corral, R. et al. Neuron 40, 65-79 (2003).
40. Novitch, B. et al. Neuron 40, 81-95 (2003).
41. Robertson, E. J. (1987) Embryo-derived stem cell lines. In Robertson, E. J. (ed), Teratocarcinoma and Embryonic Stem Cells: a Practical Approach, IRL Press, Washington, D.C., pp. 71-112.
42. Tucker et al. 2001, Nat Neurosci 4:29-37
43. Okabe, Unit 3.6 pp 1-13, in Current Protocols in Neuroscience, John Wiley & Sons 1997.
44. Weil et al. Cell Science 1998 111 2707-2715
45. Leist et al. J. Immunol. 1994 153 1778-1788

1. A method of inducing differentiation of embryonic stem (ES) cells into neuronal precursor or progenitor cells, comprising
culturing ES cells;
forming embryoid bodies (EBs);
contacting the EBs with retinoic acid (RA); and
dissociating the EBs to produce a culture of neuronal precursor cells,
wherein forming EBs comprises selecting highly proliferative ES cells and plating those cells at a measured density to form EBs.

2. A method according to claim 1, wherein the cells are plated at a density of between about 0.5×10⁵ to 5×10⁵ per ml

3. A method according to claim 2, wherein forming EBs comprises plating ES cells at a density of between about 2.5×10⁵ and 3.5×10⁵ cells per ml.

4. A method according to claim 1 wherein the EBs are maintained in non-adherent culture until dissociation of the EB cells.

5. (canceled)

6. A method according to claim 5, wherein the method comprises selecting ES cells having one or more of the following morphological features: growth in a flat monolayer; neighbouring cells not in direct contact with one another; large nuclei; many nucleoli; cells not growing on top of one another or in colony-like form.

7. (canceled)

8. (canceled)

9. (canceled)

10. A method according to claim 6 wherein the passaging is repeated at least twice in the absence of feeder cells.

11. (canceled)

12. (canceled)

13. A method according to claim 12, wherein dissociated EB cells are filtered through a mesh of about 40 μm.

14. (canceled)

15. (canceled)

16. A method according to claim 15, wherein the dissociated EB cells are plated at a density of between about 0.5×10⁵ and 2.5×10⁵ cells per cm².

17. A method according to claim 16, wherein the dissociated EB cells are plated at a density of between about 1×10⁵ and 1.5×10⁵ cells per cm².

18. A method according to any one of claim 15, comprising changing culture medium of the dissociated EB cells between about 1 and 6 hours after plating the dissociated EB cells.

19. A method according to claim 18, comprising changing the culture medium between about 1 and 3 hours after plating.

20. A method according to claim 15, wherein the dissociated EB cells or neurons are not cultured in presence of serum.

21. A method according to any one of claim 15, wherein the neuronal precursor, progenitor or neuronal cells are not cultured in the presence of growth factors.

22. A method according to claim 15, wherein the neuronal precursor, progenitor or neuronal cells are not cultured in Neurobasal medium.

23. (canceled)

24. (canceled)

25. (canceled)

26. (canceled)

27. (canceled)

28. A method according to claim 27, comprising identifying at least 90% of cells as neurons.

29. An assay method comprising determining one or more characteristics of neuronal precursor or progenitor cells or neuronal cells.

30. An assay method according to claim 29, wherein the characteristic or characteristics are one or more of neuritic growth or neurite elongation/degeneration, neuronal shape, neuronal cell death, neurogenesis, neuronal differentiation, electrical activity, synaptogenesis and/or neuronal cell markers.

31. An assay method according to claim 29, wherein the cells are produced by a method according to claim 1.

32. An assay method according to claim 31, comprising:
inducing differentiation of ES cells into neuronal precursor or progenitor cells or neuronal cells; and
determining one or more characteristics of the neuronal precursor or progenitor cells or neuronal cells under a test condition.

33. An assay method according to claim 29, comprising
culturing neuronal precursor or progenitor or neuronal cells under a first condition;
culturing neuronal precursor or progenitor or neuronal cells under a second condition;
determining or quantifying one or more neuronal characteristics of the cells; and
comparing one or more neuronal characteristics of cells cultured under the first condition with the same neuronal characteristic or characteristics in cells cultured under the second condition, respectively.

34. An assay method according to claim 33, wherein the neuronal characteristic is neurite elongation and wherein the method comprises:
quantifying levels of expression of a neurite-specific protein; and
comparing levels of expression of the neurite-specific protein;
wherein a higher level of expression under the first condition indicates that first condition increases neurite elongation.

35. An assay method according to claim 33, wherein the neuronal characteristic is neurite degeneration and wherein the method comprises:
quantifying levels of expression of a neurite-specific protein; and
comparing levels of expression of the neurite-specific protein;
wherein a lower level of expression under the first condition indicates that the first condition increases neurite degeneration.

36. A method of identifying an agent that inhibits or reduces an increase in neurite degeneration produced by a compound known to increase neurite degeneration, comprising:
culturing neuronal precursor or progenitor or neuronal cells in the presence of a test agent and under a condition known to increase neurite degeneration;
culturing neuronal precursor or progenitor or neuronal cells in the absence of the test agent and under a condition known to increase neurite degeneration;
quantifying or determining levels of neurite degeneration in the presence and in the absence of the test agent; and
comparing levels of neurite degeneration in the presence of the test agent with levels of neurite degeneration in the absence of the test agent;
wherein a lower level of neurite degeneration in the presence of the test agent compared with the absence of the test agent indicates that the agent inhibits or reduces an increase in neurite degeneration produced by or associated with the condition.

37. A method according to claim 36, wherein levels of neurite degeneration are quantified by quantifying levels of expression of a neurite-specific protein, wherein a higher level of expression of a neurite-specific protein in the presence of the test agent compared with the absence of the test agent indicates that the test agent inhibits or reduces an increase in neurite degeneration produced by or associated with the condition.

38. An assay method according to claim 33, wherein the neuronal characteristic is neuronal cell death, and wherein the method comprises:
culturing neurons under a first condition;
culturing neurons under a second condition;
quantifying or determining neuronal cell death of cells cultured under the first and under the second condition; and
comparing levels of neuronal cell death under the first condition with levels of neuronal cell death under the second condition;
wherein a higher level of neuronal cell death under the first condition compared with under the second condition indicates that the compound increases cell death; and/or
wherein a lower level of neuronal cell death under the first condition compared with under the second condition indicates that the condition reduces neuronal cell death.

39. An assay method according to claim 38, wherein the neurons express p75 neurotrophin and/or an apoptotic protein.

40. A method of identifying an agent that inhibits or reduces an increase in neuronal cell death produced by a condition known to increase neuronal cell death, comprising:
culturing neurons in the presence of a test agent and under a condition known to increase neuronal cell death;
culturing neurons in the absence of the test agent and under a condition known to increase neuronal cell death;
quantifying or determining levels neuronal cell death in the presence and in the absence of the test agent; and
comparing levels of neuronal cell death in the presence of the test agent with levels of neuronal cell death in the absence of the test agent;
wherein a lower level of neuronal cell death in the presence of the test agent compared with in the absence of the test agent indicates that the agent inhibits or reduces an increase in neuronal cell death produced by the condition.

41. An assay method according claim 33, wherein culturing under the first condition comprises culturing in the presence of a test compound or exposing the cells to a test compound, and wherein culturing under the second condition comprises culturing in the absence of the test compound or not exposing the cells to a test compound.

42. An assay method according to claim 29, for identifying a marker that indicates the differentiation state of a cell, comprising:
inducing differentiation of ES cells to produce neuronal precursor or progenitor cells; and/or
culturing neuronal precursor or progenitor cells to produce neurons;
comparing expression levels of proteins in cells at one stage of differentiation with expression levels of proteins in cells at a second stage of differentiation; and
identifying proteins whose level of expression differs in cells at the first and second stages of differentiation;
wherein a difference in expression levels indicates that the protein may be used as a marker to indicate the differentiation state of the cell.

43. An assay method according to claim 29, wherein the neuronal characteristic is synaptogenesis and wherein the method comprises measuring electrophysiological activity of the cells and/or detecting or measuring expression of one or more markers indicative of synaptogenesis.

44. A method comprising:
providing a first and a second culture of neuronal cells or neuronal precursor or progenitor cells, wherein cells in the first culture have a different genotype to cells in the second culture; and
comparing neuronal precursor or progenitor cells or neurons in the first culture with neuronal precursor or progenitor cells or neurons in the second culture.

45. A method according to claim 44, wherein cells in the first culture contain a mutation in a gene of interest, and cells in the second culture do not contain the mutation.

46. A method according to claim 44, wherein cells in the first culture contain an introduced gene, and cells in the second culture do not contain the introduced gene.

47. A method according to claim 44, wherein cells in the first culture overexpress an endogenous gene, and cells in the second culture do not overexpress the endogenous gene.

48. A method according to claim 44, comprising inducing differentiation of ES cells into the neuronal precursor or progenitor cells or neuronal cells.

49. A method according to claim 48, wherein in the first culture the ES cells contain a mutation in a gene of interest, and in the second culture the cells do not contain the mutation.

50. A method according to claim 48, comprising transfecting a first culture of dissociated EBs with a nucleic acid construct and thereby changing the genotype of cells in the first culture compared with cells in the second culture.

51. A method according to claim 44, further comprising:
culturing the first and second cultures of neuronal precursor or progenitor or neuronal cells under a test condition;
detecting, quantifying, observing or determining one or more neuronal characteristics of the cells; and
comparing the neuronal characteristics of the cells in the first culture with the neuronal characteristics of the cells in the second culture.

52. A method according to claim 51 wherein culturing under the first condition comprises culturing the cells in the presence of Aβ peptide and culturing the cells under the second condition comprises culturing the cells in the absence of Aβ peptide.

53. A method according to claim 52, wherein the neuronal characteristic is neurite degradation.

54. A method substantially as herein described.
